(19) United States
(12) Patent Application Publication
(10) Pub. No.: US 2025/0371713 A1
(43) Pub. Date: Dec. 4, 2025

(54) Mapping glutathione deficiency and/or dysfunction of mitochondria

(71) Applicant: Debra Strick Rivera, Topanga, CA (US)

(72) Inventor: Debra Strick Rivera, Topanga, CA (US)

(21) Appl. No.: 18/675,621

(22) Filed: May 28, 2024

(51) Int. Cl. G06T 7/00; A61B 5/055; A61K 49/14; A61K 49/18

(52) U.S. Cl. CPC G06T 7/0016; A61B 5/055; A61K 49/14; A61K 49/1812; G06T 2207/10088; G06T 2207/20224; G06T 2207/30016

(57) ABSTRACT

The disclosed invention is a method for mapping glutathione deficiency and/or mitochondria dysfunction in disease states, utilizing glutathione and its precursors as imaging tracers in conjunction with medical imaging techniques, particularly magnetic resonance imaging. The innovation involves administering glutathione or glutathione-increasing interventions, such as oral liposomal reduced glutathione, to enhance imaging accuracy. The proposed methodology aims to identify and monitor regions of depleted reduced glutathione (GSH) in various organs and tissues. The maps generated through this process provide valuable biomarkers for toxic exposure, early biological effects, and health risks. The approach is versatile, offering applications in mental health, neurological disorders, inflammation, organ dysfunction, and personalized treatment and risk assessments.

### CROSS REFERENCE TO RELATED APPLICATION

Cross-reference to application #XXXXXXXXXXX.

### STATEMENT REGARDING FEDERALLY SPONSORED RESEARCH OR DEVELOPMENT

Not Applicable.

### FIELD

The present invention proposes the administration of a glutathione increasing intervention, including but not limited to oral liposomal reduced glutathione, as an imaging tracer for mapping depleted reduced glutathione (GSH) pools in order to locate tissues with mitochondrial dysfunction. Such a measure of glutathione-uptake can also be enhanced by pairing the glutathione or glutathione precursor with a signal or contrast enhancing substance for medical imaging.

### BACKGROUND

Glutathione is a tripeptide in reduced form, comprising of glycine, glutamate, and cysteine, that has a fundamental role in all eukaryotic cells of maintaining the homeostasis of mitochondria and buffering the redox balance of the tissue. All biological stressors lead to increased reactive oxidative species that are ameliorated by reduced glutathione stores, including but not limited to exogenous toxin exposure, stress, blunt trauma, infection, sleep disturbance, cardiovascular events, and glucose spikes. Mitochondrial dysfunction is gaining momentum as a common biomechanism across diseases, including neurodegeneration, mental illness, cardiovascular disease, infection, brain injury, cancer proliferation and drug resistance, organ failure, and metabolic maladies.

There are diverse ways in which endogenous glutathione stores can be replenished and supported. For example, U.S. Pat. No. 11,730,711 B2 from mid 2023 presents the use of NAC for increasing endogenous glutathione levels. Life-style interventions such as diet, exercise, improved sleep, and other stress management techniques can replenish depleted glutathione stores. Reduced glutathione is up-taken, even against a gradient, into the areas in which the reduced glutathione stores are most depleted.

Whether given orally or intravenously, it is difficult to deliver reduced glutathione across the blood brain barrier (10.1254/jphs.08R01CR). However as described by U.S. Pat. Nos. 8,349,359B2 and 8,349,359B2, encasing the reduced glutathione in a liposome enhances bioavailability. Liposomes are capable of crossing the blood brain barrier, and can be non-polar, or charged, and can also be augmented with peptides, antibodies, or polyethylene glycol (10.2147/IJN.S117210). Japanese Patent JP6267735B2R describes the use of glutathione or glutathione uptake ligands embedded in pegylated liposomes to further enhance crossing of the blood brain barrier.

Glutathione can be reliably and repeatably measured in vivo using magnetic resonance spectroscopy. Glutathione measurements using magnetic resonance spectroscopy have been conducted in neurological and psychiatric disorders (e.g., 10.1002/jmri.25356, 10.1002/jmri.24970). The majority of studies measure the glutathione in a single or set of individual voxels. It is the rare approach that allows for chemical shift mapping of glutathione, in which an array of spectroscopic voxels are collected, for example in 10.1002/mrm.27702. A 2021 article (10.3390/antiox10091407) details 64 published studies of glutathione magnetic resonance spectroscopy of the living human brain. The concentration of glutathione can be derived and compared across brain regions and has yielded statistically significant differences between groups of individuals with no cognitive impairment, mild cognitive impairment, and Alzheimer's Disease (10.1002/hbm.24799). In another demonstration, the glutathione levels of non-sedated children were characterized (10.3174/ajnr.A5457). In another study, the glutathione levels were shown to be stable in 5 brain regions across a 4-year longitudinal study showing decreases in glutathione in the anterior cingulate cortex in patients with refractory psychosis compared to controls using a 7 T magnet (clinical MRI is typically at 1.5 T or 3 T) (10.1038/s41380-023-01969-5). In a cohort of patients with neurocognitive deficit post COVID-19 infection, a statistically significant reduction of glutathione in the grey matter was observed in the frontal cortex as compared to age-matched healthy controls using a proton-edited glutathione mapping sequence (10.1515/nipt-2022-0006). US Patent US 2021/0128509 A1 proposes a method by which dosing of intranasal delivery of the glutathione precursor N acetyl cysteine (NAC) is informed through measurement of glutathione using magnetic resonance spectroscopy (MRS) after dosage. Such a method specifies that this technique is only in the context of titration of intranasal NAC dose, rather than in the context of administering any other intervention or informing disease status.

While glutathione metabolic imaging with magnetic resonance spectroscopy is possible, the overlap of the metabolite with other species of greater abundance can lead to complexity and inaccuracy. However, methods exist by which contrast or signal can be enhanced for diverse imaging modalities. Chinese Patent CN102914570B describes use of a small magnetic sphere, nanogold, and thionine modified carbon composite electrode to amplify the electrochemical detection of the glutathione system, recognizing the utility of glutathione measures, although in an hitherto unrelated approach. Positron emission tomography tracers for targeting the ROS detox pathways exist, for example the protein channel responsible for bringing cysteine into the cell which is then converted into glutathione (10.1186/s12974-018-1080-1). Another example is the U.S. Pat. No. 6,217.849 in which the use of liposomes containing contrast enhancing substances for computed tomography imaging are administered. The use of microbubbles as a contrast agent in ultrasound, as well as ultra high field magnetic resonance imaging is known, including implementations in which the microbubble contains a ferromagnetic nanoparticle as in US20220265869A1. While as previously mentioned, it is feasible to place glutathione, glutathione analogues, or glutathione precursors onto PEG microbubbles, this is also not yet a documented technique for imaging or mapping glutathione deficiency. Nor has the use of tagging glutathione with a magnetic susceptibility alternating substance been described in prior art. Ergo, while there are numerous methods by which glutathione imaging could be enhanced, the leap of administering glutathione or glutathione-enhancing interventions as a means of mapping mitochondrial dysfunction and/or glutathione deficiency has not yet been described.

### BRIEF DESCRIPTION OF THE INVENTION

Herein we propose the use of magnetic resonance imaging of glutathione to inform metabolic status and/or resilience of tissue to impending stress. In order to overcome the high-variability of baseline glutathione levels across individuals, we map glutathione uptake by administering glutathione or one or more glutathione increasing interventions to highlight areas in which GSH is depleted. By so doing, we invert what is typically a measure of depleted glutathione, into an increase in uptake. Glutathione and glutathione uptake measures have the potential to act as biomarkers of toxic exposure, early biological effects, and health risk. As a surrogate to classical endpoints such as survival or disease incidence, glutathione and glutathione uptake measures have the potential to enhance tailored treatments and treatment development for chronic and acute disease. By administering a substance that increases glutathione, an imaging experiment using the subtraction method can be conducted in which the altered condition is compared to the baseline condition.

As a first exemplary embodiment, please consider a study in which a participant drinks a liposomal preparation of glutathione and lies down in a Magnetic Resonance Imaging Scanner. It takes approximately 5 minutes to get a person into the scanner and measure and correct for imperfections in the static magnetic field of the scanner (e.g., 3 tesla (T) or 7 T) superconducting magnet. Over the course of the remaining hour, several proton chemical shift imaging maps are collected using either conventional or edited magnetic resonance spectroscopic imaging techniques. The change of signal over time allows maps that highlight regions in which glutathione is depleted. These maps of rate and overall change serve as a biomarker that informs the vulnerability and resilience of the tissue regions. In a related embodiment, the oral glutathione can be given after a baseline glutathione spectroscopic measurement is conducted and while the person is still in the scanner in the event that a true baseline measure is preferred.

In a further embodiment, the maps described in the prior embodiment, can be used to infer brain regions of mitochondrial dysfunction and thereby allow for diagnosis or classification of mental health of neurological disorders to enhance treatment strategy. In one such embodiment, a patient with Parkinson's Disease is administered with intravenous NAC (as in 10.1097/WNF.0b013e31829ae713) in order to determine the brain region or regions in order to facilitate planning for treatments including but not limited to deep brain stimulation, non-invasive stimulation or lesioning, or medical or dietary interventions. In another embodiment, the presence of low glutathione at baseline in the frontal cortex grey area (as was seen in 10.1515/nipt-2022-0006), or areas of maximum glutathione change after administration of injected NAC or orally administered liposomal glutathione, can be used to identify and/or monitor treatment of individuals suffering from post-acute neurocognitive symptoms following a COVID-19 infection.

In yet a further embodiment, the maps can be used to identify the source of blood or serum markers associated with infection, inflammation, or organ failure, when standard methods are inconclusive. For example, an individual can be administered microbubbles with a glutathione analog on the outer surface, to map the kinetics of glutathione uptake in the abdomen in order to determine the area from which the source of an inflammatory serum marker is originating. Such an approach can be applied to the heart, lungs, liver, kidneys, stomach, or other organs in the body.

The herein described approach can be used in a series of embodiments in order to determine the efficacy of a given intervention in a single individual or in a group of individuals. As baseline values of glutathione, or the ratio of reduced to oxidized glutathione can very across individuals, such an approach of characterizing the change from baseline in a given individual can overcome the confounding variable of intra-individual variation in baseline. In one embodiment, one or more individual is given a dietary supplement derived from cruciferous vegetables, sulforaphane. Prior to being given the supplement, the participant(s) undergo glutathione-uptake mapping as has been described above, using intranasal NAC or glutathione. After a period of time, e.g., 6 weeks, the participant(s) return to repeat the glutathione-uptake mapping procedure to identify regions in which the sulforaphane dosage was sufficient to overcome glutathione deficiency. If there is no change in glutathione uptake, it can be inferred under the assumption that no other variables were altered (e.g., no change in stress level, sleep quality, exercise, diet, infection, medication, etc.) that the sulforaphane was not effective for this dose and duration for this individual. On the other hand, if the uptake maps indicate that one or more areas uptake less glutathione and the aforementioned assumption holds, it can be inferred that the glutathione deficiency in those regions has been reduced by the sulforaphane regiment. If some regions do not show reduced uptake, an increase in dose and/or duration of the sulforaphane regiment, or supplementation with additional interventions, including but not limited to dietary or life-style changes, may result in further improvements. Further embodiments replacing sulforaphane with life-style, pharmaceutical, dietary/nutraceutical, or even public policy, environment, or services can be imagined.

In yet another embodiment, we can apply the approach of deuterium labeled MRI, as has been done for example in deuterium labeled glucose (10.3389/fimmu.2023.1258027), to the glutathione system. See for example prior art US 2020/0385342 A1, wherein intranasal preparations of NAC related compounds are deuterated with the ³H isotope, which is not detectable with MRI. However, modifying the process described in US 2020/0385342 A1 by using the ²H deuterium isotope, we then have a substance that can be identified with no background signal with a magnetic resonance imaging scanner. This allows for straightforward imaging of for example glutathione uptake or cysteine (e.g., administered in the form of deuterated NAC amide) into regions for which glutathione is depleted.

In a further embodiment, individuals identified as high risk for Alzheimer's Disease, including but not limited to those carrying two copies of the apolipoprotein E gene APOE4, can be continually monitored for pre-symptomatic tissue changes that precede beta amyloid plaque formation using glutathione-uptake mapping.

In yet another embodiment, glutathione uptake in a given individual and or a specific anatomical location can be compared to the mean and standard deviation of age matched individuals to inform health risk. For example, a method by which the hippocampus, prefrontal grey matter, or heart glutathione-uptake measure as normalized by the chronological age informs risk of a given individual, or speaks to health disparity across a population. Glutathione-uptake measurements can also be used as a means of identifying individuals most likely to benefit from a given therapy to treat glutathione deficiency. For example, it has been shown that NAC supplementation only improves exercise performance in those demonstrated to have low levels of glutathione (10.1016/j.freeradbiomed.2017.12.007).

### BRIEF DESCRIPTION OF THE DRAWINGS

FIG. 1 details various factors that can lead to the depletion of glutathione levels in the body.

FIG. 2 depicts the harmful effects of reactive oxygen species (ROS) accumulation within the body, highlighting diseases and aging processes associated with redox imbalance including but not limited to oxidative stress.

FIG. 3 shows an example proton spectroscopy MEGA Press baseline spectra 1, spectra post-administering a glutathione-increasing intervention 2, and a subtraction spectra 3, with an arrow depicting the primary glutathione peak 4.

FIG. 4 shows a rendering of a chemical shift imaging spectra at baseline 5.

FIG. 5 shows a rendering of a chemical shift imaging spectra after administering a glutathione increasing intervention 6.

FIG. 6 shows a rendering of the subtraction chemical shift image 7 obtained by subtracting the baseline and post-intervention chemical shift imaging signals.

FIG. 7 is a depiction of projecting the chemical shift imaging subtraction image 7 onto an anatomical image 8.

FIG. 8 is a workflow diagram describing a set of steps used to obtain glutathione uptake images with a baseline measurement taken at 5 minutes after administering the intervention.

FIG. 9 is a workflow diagram describing a set of steps used to obtain glutathione uptake images with a baseline measurement taken before administering the intervention.

FIG. 10 is a workflow diagram of extracting heat maps from glutathione uptake imaging and projecting the image onto an anatomical map.

FIG. 11 is a workflow diagram for a method in which tagged or contrast enhancing glutathione is administered and imaged.

FIG. 12 is a workflow diagram for evaluating changes in glutathione in response to an intervention.

### DETAILED DESCRIPTION OF THE DRAWINGS

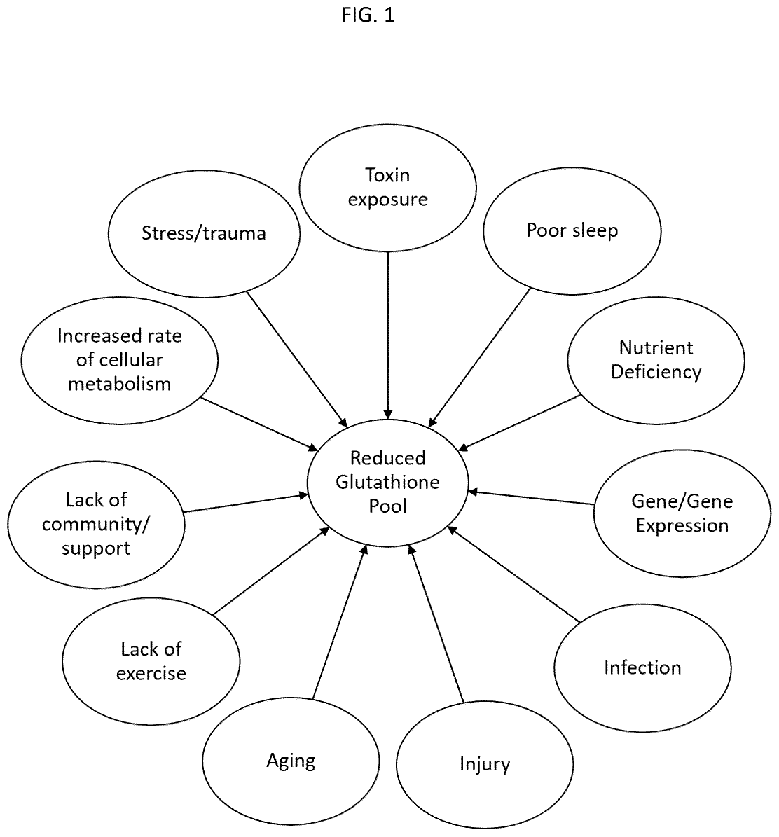

FIG. 1 enumerates various factors that lead to glutathione depletion, including poor diet, pollution exposure, chronic stress, poor sleep, lack of community, genetic predisposition, and infections.

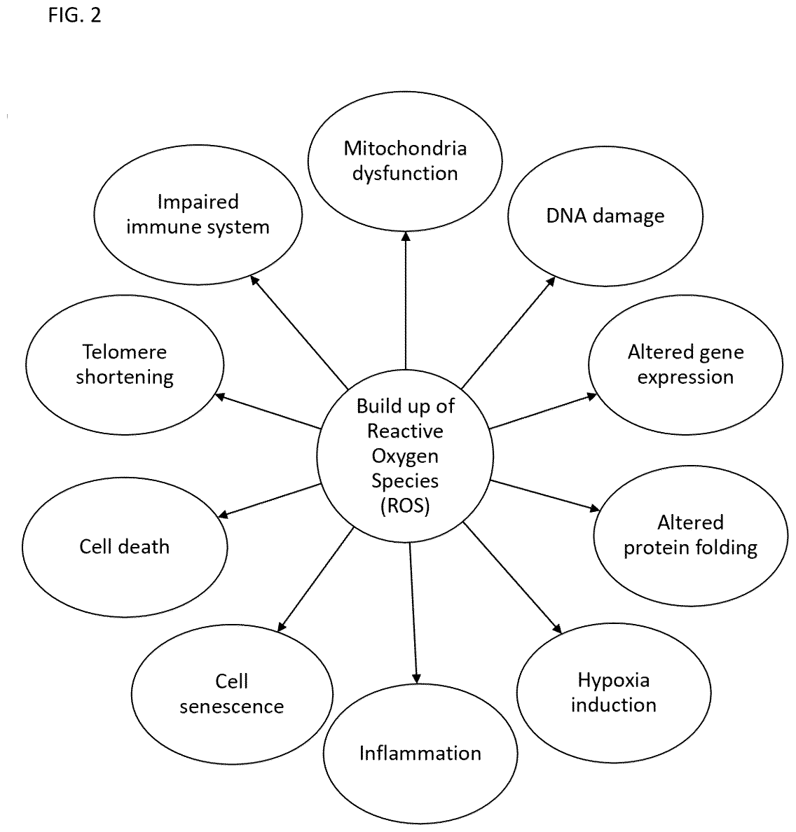

FIG. 2 depicts the harmful effects of reactive oxygen species (ROS) accumulation within the body, as a result of depleted glutathione. A build up of reactive oxygen species leads to oxidative stress, which triggers inflammation, hypoxia, and glycolysis. Oxidative stress also results in telomere shortening, nuclear and mitochondrial DNA damage, altered methylation and gene expression, altered protein folding, mitochondrial damage, and cell senescence. An extreme build up of oxidative species will trigger cell death, including ferroptosis. Even further build up will lead to necrosis.

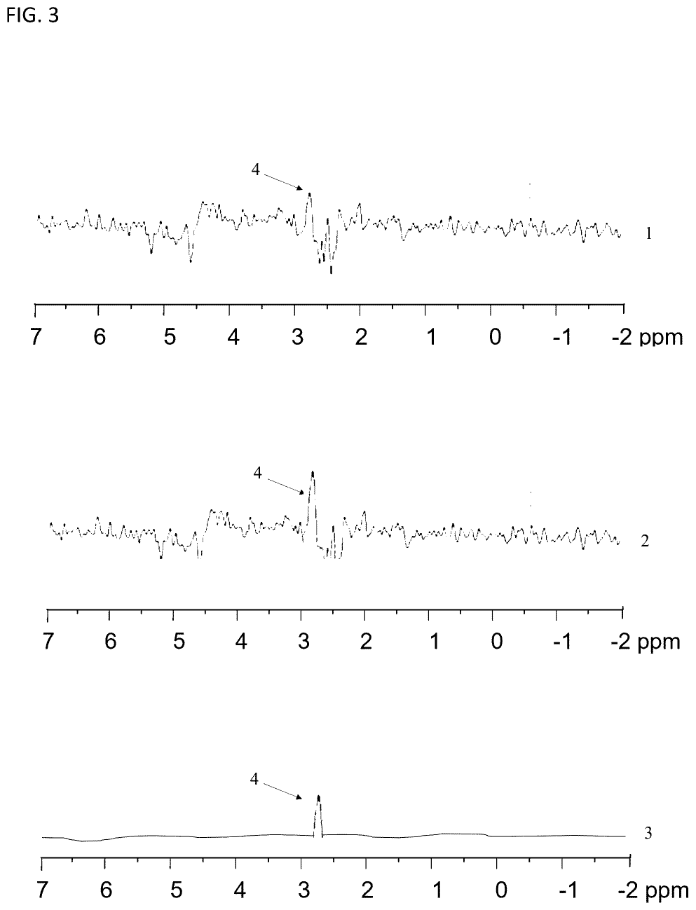

FIG. 3 shows an example of characteristic signals from proton spectroscopy MEscher-GArwood-Point-RESolved (MEGA Press) spectroscopy for use with glutathione uptake imaging. Such a sample is representative of a 2.5 cm isotropic volume, termed a voxel. An appropriate set of sequence parameters includes but is not limited to a 3 Tesla magnetic resonance scanner, repetition time of 2500 ms, echo time of 120 ms, bandwidth of 2500 Hz, a 180-degree refocusing pulse at 4.4 ppm, water suppression, and a total scan time on the order of 15 minutes. Alternatively, although not depicted, an editing spectra approach can be used. A baseline spectra 1 is obtained, as depicted in the top row. Next, after a glutathione increasing intervention, or exogenous glutathione is administered, a post-intervention spectra is obtained 2 (middle row). A subtraction spectra 3 (bottom row) is calculated by subtracting the baseline spectra 1 from the post-intervention spectra 2. An arrow depicting the primary glutathione peak 4 appears in all of the spectra.

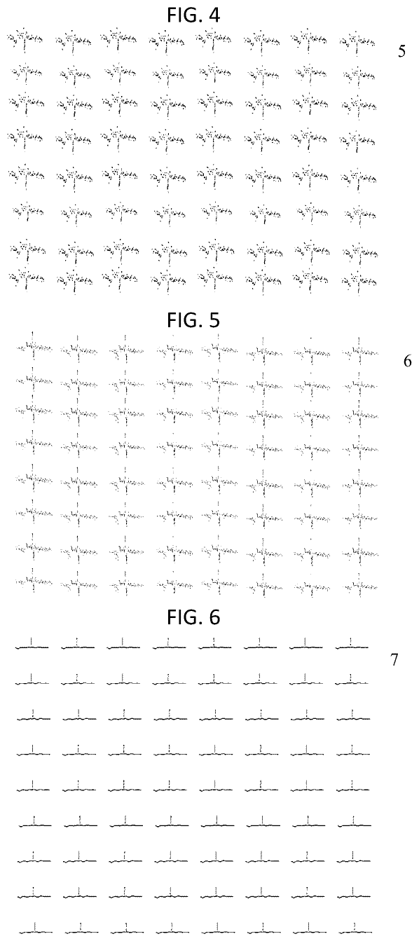

FIG. 4 demonstrates a 2-dimentional matrix of chemical shift imaging spectra. A chemical shift imaging matrix can be either two or three dimensions. The presented image is to indicate a chemical shift imaging matrix of a baseline spectra 1, depicted as an array, and thus a chemical shift imaging spectra array at baseline 5.

FIG. 5 is demonstrates a 2-dimentional matrix of chemical shift imaging spectra. A chemical shift imaging matrix can be either two or three dimensions. The presented image is to indicate a chemical shift imaging matrix of a post intervention spectra 2, depicted as an array, and thus a chemical shift imaging spectra array after uptake of glutathione or after an increase of glutathione as a result of a glutathione-increasing intervention 6.

FIG. 6 shows a rendering of the subtraction chemical shift image 7 obtained by subtracting the baseline chemical shift imaging matrix 5 from the post-intervention chemical shift imaging signal 6. By using the subtraction technique the complexity in the signals is reduced, allowing for mapping rather than single-volume imaging of glutathione.

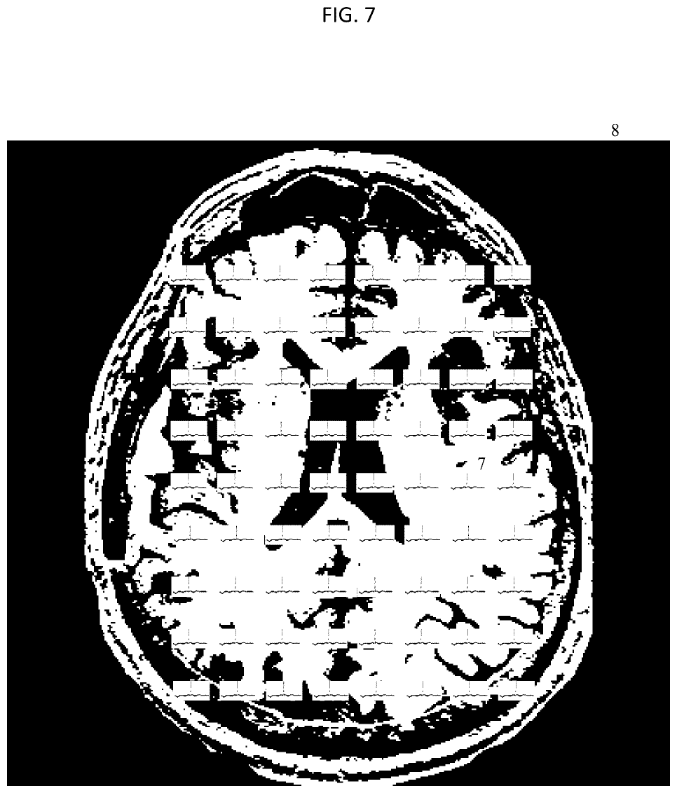

FIG. 7 is a depiction of projecting the chemical shift imaging subtraction image 7 onto an anatomical image 8. By projecting the chemical shift subtraction image onto an anatomical map of the corresponding regions from which the signal is collected allows for mapping.

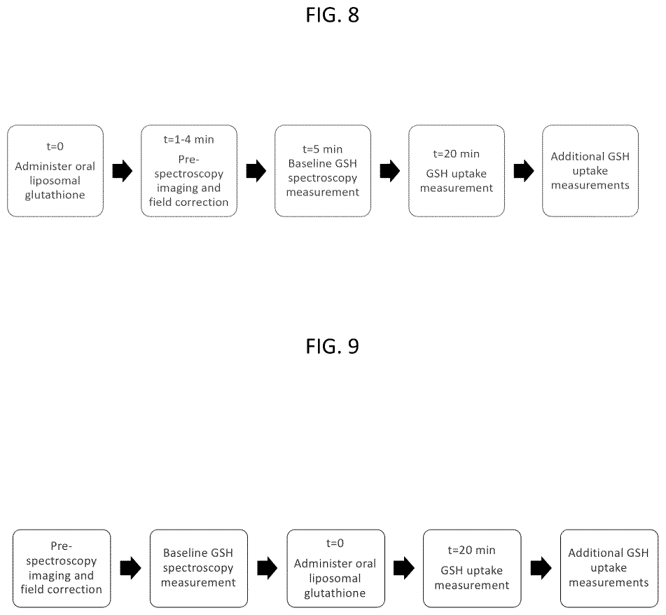

FIG. 8 is a workflow diagram describing a set of steps used to obtain glutathione uptake images using magnetic resonance spectroscopy with a baseline measurement taken at 5 minutes after administering the intervention. In this case, the subject is administered the glutathione increasing substance immediately before the subject is placed in the magnetic resonance scanner. Next the static-magnetic field is mapped and corrected. Power adjustments may also be necessary depending on the field strength. After the scanner settings and anatomical localizer images are collected. In total, these preparations take less then 5 minutes to complete. The baseline spectroscopic image begins at 5 minutes, and the uptake images begin circa 20 minutes after dosing. Additional measurements may also be collected for further data points to capture uptake kinetics on a molecular level as well as anatomical distribution.

FIG. 9 is a workflow diagram describing a set of steps used to obtain glutathione uptake images with a baseline measurement taken before administering the intervention. In this case, the subsect enters the scanner and undergoes the field adjustments, power adjustments, and anatomical images, followed by a true baseline measurement. Next, the subject administered the intervention. If the subject is removed from the scanner to administer the intervention, field adjustments, power adjustments, and anatomical images are repeated. After sufficient time for the uptake to occur, the spectroscopic images for the post-administered data collection commences.

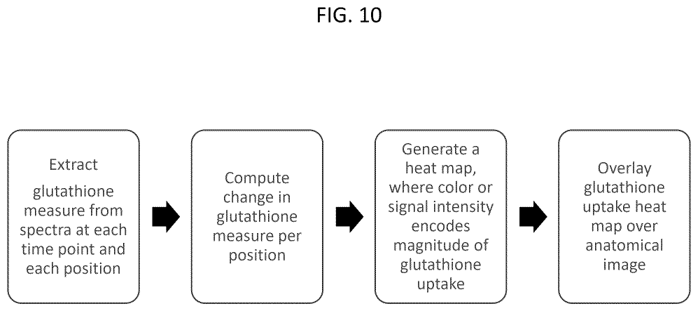

FIG. 10 is a potential workflow diagram of data processing steps to extract heat maps from glutathione uptake imaging and projecting the image onto an anatomical map. First the glutathione levels are calculated from each location in each time point of the spectra. The level can be calculated as concentration or from characteristics of the estimated main peak of glutathione. Next, the uptake is computed by evaluating the difference in the heights at different time points. This allows for the optional step of normalizing the change in glutathione level by the glutathione level at the latest time point. The alternative approach of estimating uptake by first calculating the subtraction spectra does not require calculating the level at a single time point. Once the uptake information is calculated, it is encoded as signal intensity or color as a heat map. In the final step the heat map is projected onto an anatomical image to inform spatial location.

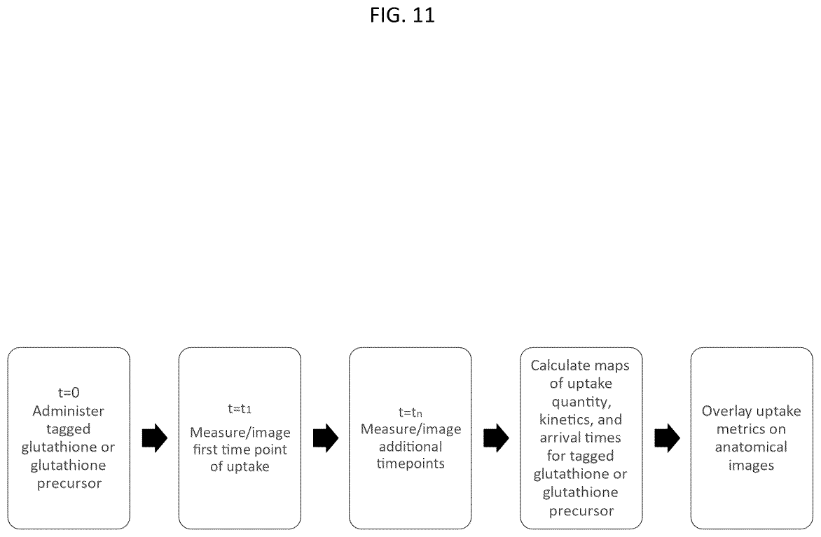

FIG. 11 is a workflow diagram for a method in which tagged or contrast enhancing glutathione is administered and imaged. As a first step, the tagged or contrast-enhanced glutathione preparation is administered at time 0. At subsequent time points, images are collected. After collecting the images, the uptake levels, kinetics, and locations are computed and translated into heat maps. In the final step, one or more kinetic maps are projected onto anatomical images.

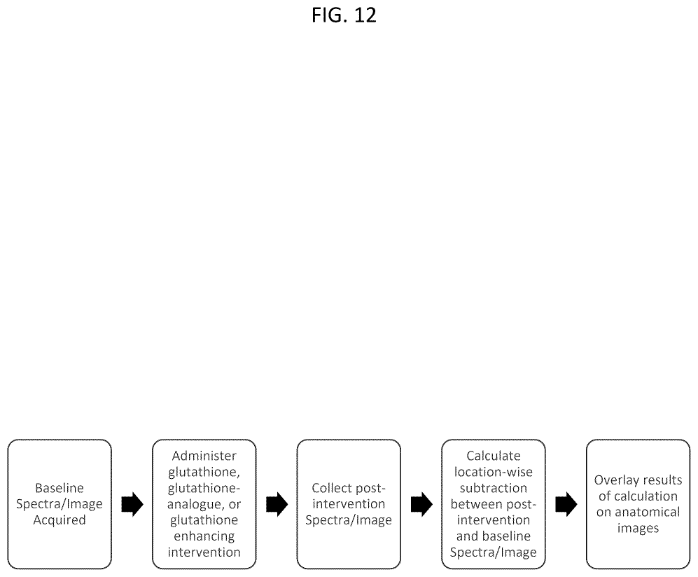

FIG. 12 is a workflow diagram for evaluating changes in glutathione in response to an intervention. The initial baseline image is acquired, and this need not be on the same day as the subsequent images. The intervention is then initiated and it may last for days, weeks, or months. Subsequently, the uptake image is acquired. The change in glutathione from the post-and pre-intervention images is calculated, translated to heat maps, and projected on an anatomical image.

The herein described embodiments and figures are not an exhaustive description, but rather presented to help elucidate the utility of the claims. All presented features can be used in combination or in isolation, and again are examples rather than a complete elaboration of the scope and applications of the set of claims that define the invention.

What is claimed for:

1. A method for localizing, mapping, or monitoring regions of interest in a medical image comprising administered glutathione and/or a glutathione-increasing intervention and acquiring the glutathione signal with an imaging modality including but not limited to magnetic resonance spectroscopy.

2. The method of claim 1, wherein the glutathione-increasing intervention is administered orally as reduced glutathione in pure or modified form contained within a liposome.

3. The method of claim 1 in the signal of interest is the difference of the glutathione present before and after the administration of the exogenous glutathione or glutathione-increasing intervention, comprising the steps of:
a) obtaining an image prior to the uptake time-wind of the administered intervention,
b) administering the glutathione or glutathione-increasing intervention,
c) waiting for the administered intervention to take effect,
d) obtaining one or more images after the administered intervention takes effect,
e) computing the difference image between the images taken before and after administering the intervention,
f) and optionally projecting the results onto an anatomical image.

4. The method of claim 2 in which exogenous glutathione or a glutathione-increasing species is bound to or accompanied by a magnetic susceptibility altering moiety.

5. The method of claim 1 in which the glutathione and/or glutathione increasing species is administered intravenously or via injection.

6. The method of claim 1 in which the glutathione increasing intervention is
i) a source of cysteine administered by any route, including but not limited to N-acetyl cysteine or whey protein;
ii) an up-regulator of Nrf2;
ii) or a down-regulator of oxidative species.

7. The method of claim 3 wherein the glutathione and/or glutathione-increasing species is applied intranasally.

8. The method of claim 3 wherein the baseline image is taken on a day that proceeds measures acquired on one or more subsequent days.

9. The method of claim 8 in which the glutathione increasing intervention is a lifestyle intervention, including but not limited to one or more of the following: sleep changes, exercise, therapy, bodywork, acupuncture, hypnosis, meditation, a group or individual activity, dietary change, a ritual or ceremony, stress management technique, change in community or environment, sauna, taking of vitamins or supplements, virtual experience, and or any combination of the aforementioned life style interventions.

10. The method of claim 8 in which the intervention is a medical intervention including but not limited to one or more of the following: vagus nerve stimulation, intravenous gamma globulin, antihistamine, hormone therapy, or stem cell administration.

11. The method of claim 1 in which the measure is used to identify areas of inflammation, neuroinflammation, mitochondrial dysfunction, neurodegeneration, trauma, injury, or a region of reduced resilience to stressors.

12. The method of claim 1 in which the method is used to locate, diagnose, treat, or monitor cellular metabolic status, organ dysfunction including but not limited to the liver, heart, eye, retina, lungs, placenta, intestines, and kidneys, cardiovascular disease and/or risk, neurodegeneration, neuroinflammation, mental illness, tissue damage or injury, inflammation, pain, immune response, insulin insensitivity, glucose metabolism alterations, and mitochondrial dysfunction.

13. The method of claim 11 in which the region or regions of interest are used to classify, treat, or monitor mental illness or neurological impairment.

14. A method in which magnetic resonance imaging and/or spectroscopy of glutathione is used to map disease for localization, diagnosis, treatment guidance, treatment monitoring, and/or evaluating the efficacy of a treatment in one or more individuals.

15. A method as in claim 13, in which the maps are paired with measures of the ratio of reduced to oxidized glutathione in biological samples from the same individual.

16. A method as in claim 13 further specified in that the glutathione measured non-invasively can be either exogenous supplied glutathione or the endogenous forms of glutathione including: reduced glutathione, total glutathione, oxidized glutathione, and/or the ratio of reduced to oxidized glutathione.

17. The method as in claim 15 in which the metric or map is used to map disease and/or inform health or tissue or an individual, redox status, inflammation, autoimmune or immune reactions, autonomic nervous system dysfunction, mast cell activation, arthritis, allergic reaction, mitochondrial health/dysfunction, and or biological age of an individual or a given tissue region.

18. The method as in claim 1 in which the glutathione or glutathione-increasing intervention is administered via injection, intravenously, or via absorption from the buccal mucosa.

19. The method as in claim 1 in which the glutathione, glutathione analogue, or a glutathione-increasing intervention is combined with an imaging signal or contrast enhancing moiety, substance, or substitution, including but not limited to an unpaired-nuclei deuterium isotope for magnetic resonance imaging, microbubbles for CT or ultrasound, or a radioactive tracer for PET scan.

20. A method comprising administering a glutathione-enhancing intervention and mapping the metabolic changes associated with increased reduced glutathione to oxidized glutathione ratio, such as an increase in Krebs cycle products, a reduction in glycolysis products, a decrease in Krebs cycle reactants, an increase in glycolysis reactants, or ratios there of.
